(19) United States
(12) Patent Application Publication
(10) Pub. No.: US 2005/0107728 A1
(43) Pub. Date: May 19, 2005

(54) Therapeutic socks

(76) Inventors: Kurt M. Vetters, Greenfield, IN (US); James G. Spahn, Indianapolis, IN (US); James D. Spahn, Indianapolis, IN (US)

Correspondence Address: Clifford W. Browning, Suite 3700, 111 Monument Circle, Indianapolis, IN, 46204-5137, US

(21) Appl. No.: 10/990,738

(22) Filed: Nov. 17, 2004

Related U.S. Application Data
(60) Provisional application No. 60/520,566, filed on Nov. 17, 2003.

(51) Int. Cl. A61F 5/00

(52) U.S. Cl. 602/23

(57) ABSTRACT

A therapeutic sock, comprising a sock having a first end to receive toes, a second end to overlay a calf, and a heel zone therebetween to overlay a heel; and an elevating component shaped to contour the calf and to elevate the posterior heel, ankle and sides of a foot when in use, and positioned within and attached to the sock between the heel zone and the second end whereby when the sock is in use the elevating component is positioned over the Achilles tendon and relieves pressures caused by direct contacts with any and all surfaces.

This application claims the benefit of co-pending U.S. provisional patent application Ser. No. 60/529,566, filed Nov. 17, 2003.

This invention relates to pressure relief and comfort for the feet and ankles of individuals who have the ability to ambulate.

### BACKGROUND OF THE INVENTION

Basic research and clinical findings have demonstrated the difficulty in preventing and treating pressure ulcers of the lower extremities. The heel and foot have numerous bony prominences that predispose this area to the mechanical effects of wedge force amplication due to the shape of these bony prominences. The combination of the bony prominence wedge effect, lever action of the leg itself, poor vascularity and stiff viscolelastic soft tissue creates a most difficult situation when a person is placed in a recumbent position.

Understanding the vertical shearing that occurs with the force amplification associated with the wedge shape of the bony prominences and its detrimental effect on already poorly vascularized soft tissue explains the difficulty in preventing and treating soft tissue breakdown in and about the foot.

The problems of foot drop, lateral rotation of the foot, and calf distortion must also be addressed. In short, the prevention and treatment of lower extremity soft tissue necrosis must include mechanical stress relief, maintenance of proper calf configuration, and proper lower extremity and foot positioning. The ability to manage these factors along with the hazardous wedge effect of the numerous bony prominences in the foot-ankle complex is necessary in order to protect the soft tissue of the lower extremities from the detrimental mechanical stress forces occurring while an individual is in a recumbent position.

Pressure ulcers “ischemic necrosis” of the ankle-heel-foot anatomical region of the body (henceforth known as the “region”) accounts for approximately 50 percent of the pressure ulcers recorded in the post acute care facilities. The physiologic reason for this problem is that the impaired hemodynamic condition of the convalescent patient is accentuated when the patient is placed horizontally on a support surface. Combined with this inherent problem is the adverse effect caused by the fulcrum force amplification secondary to the length of the leg and the force amplification secondary to the wedged-like shape of the numerous bony prominences of the region.

This combination creates a severe risk for ischemic necrosis of the soft tissue in and about the posterior, side and upper area of this region. The soft tissue has no tolerance to endure the load (weight) placed on it when this region is supported by a support surface.

There is a lack of devices that will help unload the soft tissue at risk and yet allow ambulization with assistance. There are a number or heel protectors and heel elevators in the prior art, but none adequately satisfy the needs of the patients and care-givers. Most low-end heel protectors “cup” the heel with material such as air/foam/gel for pressure relief, but this process does not relieve pressure because it is simply substituting a surface for the heel to be placed.

Because other products in this market tend to “cup” the heel, they actually apply pressure to the area or areas of the heel and foot causing further skin damage. The foam cup incased in a variety of materials have a tendency to block air circulation necessary for healing and for patient comfort.

Relatively low-price heel elevators ($12-$40 range) generally include open-cell foam blocks that fit over the heel. The type of open-cell foam used in these products often cause skin irritation, skin shear, chaffing, pressure sores or pressure ulcers because they often collapse, forming a rigid surface that is in direct contact with the heel.

In addition to the cupping of the heel and lack of ability to ambulate, such products often have an elastic band like that of a sock that can cause irritation on the leg and effect vascular circulation.

### SUMMARY OF THE INVENTION

The therapeutic sock of the present invention reduces the pressure of the heel of a patient who would be in a reclined position who is at risk of skin breakdown or irritation. In addition to the heel, the invention also protects both medial and lateral malleolus on the sides of the foot. By unloading the pressure of the heel and reducing impact with the bed sheet or other surface, incidence of skin damage/irritation is greatly reduced.

The therapeutic sock of the present invention is designed to unload the region while allowing ambulization without removing or adapting the device to do so.

The therapeutic sock of the present invention is especially beneficial for use by the following types of persons:
a. Hospitalized patients who have come out of surgery with heel shear (heel has rubbed on the operating room table during the surgical procedure).
b. Hospital, rehabilitation center, nursing home and home care patients who spend a large amount of time in bed, but who are ambulatory. These patients are susceptible to developing skin shear, pressure sores, burning heels and pressure ulcers on any portion of the foot, especially the heel.
c. Persons with diabetes who are at risk of developing diabetic foot ulcers caused by this disease.
d. Persons with poor circulation who develop ulcers as a result of circulatory impairment.
e. Persons with injury to the bones in relation to the foot and ankle region, such as surgery or blunt trauma.

Therefore, it is an object of the invention to provide a heel/ankle “unloading” device that functions as a system to help reduce the incidence of chaffing, shin shear, pressure sores or pressure ulcers, or diabetic ulcers.

It is another object of the invention to provide a heel/ankle “unloading” device that is easy to apply and remove from the lower leg of a patient.

It is another object of the invention to provide a heel/ankle “unloading” device that protects the heel/ankle and both medial and lateral malleolus area of the patient regardless of his/her position-whether lying on the back, right side or left side.

It is another object of the invention to provide a heel/ankle “unloading” device that can be machine washed and tumbled or drip dried as one complete unit.

It is another object of the invention to provide a heel/ankle “unloading” device that does not include separate parts that can be misplaced or lost.

It is another object of the invention to provide a heel/ankle “unloading” device that remains in its proper secured position on the lower leg when the patient moves or is turned from side to side.

It is another object of the invention to provide a heel/ankle “unloading” device that significally reduces pressure to the heel/ankle by relieving pressure from the region, even when placed for long periods of time.

It is another object of the invention to provide a heel/ankle “unloading” device that does not restrict blood flow to, or air circulation around, the foot of the patient.

It is another object of the invention to provide a heel/ankle “unloading” device that adjusts to fit a wide range of sizes.

It is another object of the invention to provide a heel/ankle “unloading” device that minimizes friction and reduces pressure at the point of contact on the heel/ankle and both medial and lateral malleolus area, thereby reducing the possibility of skin shear, chaffing or pressure sores to these areas.

It is another object of the invention to provide a heel “unloading” device that can be used on a patient both when in bed, a sitting position, or ambulating.

It is another object of the invention to offload pressure by raising the area between the heel and calf, which reduces the load of the heel, while providing the ability to ambulate.

### BRIEF DESCRIPTION OF THE DRAWINGS

FIG. 1 is a bottom view of a preferred embodiment of the therapeutic sock of the present invention.

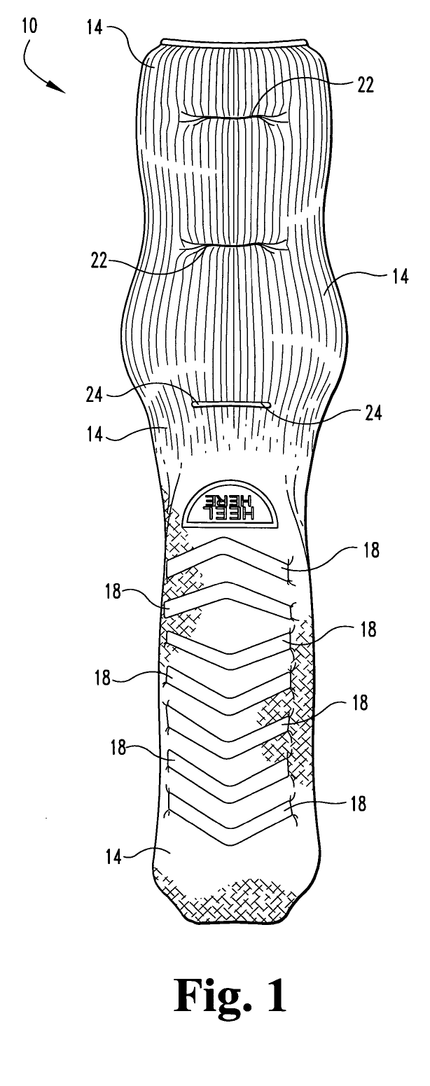

FIG. 2 is a top view of the therapeutic sock of FIG. 1.

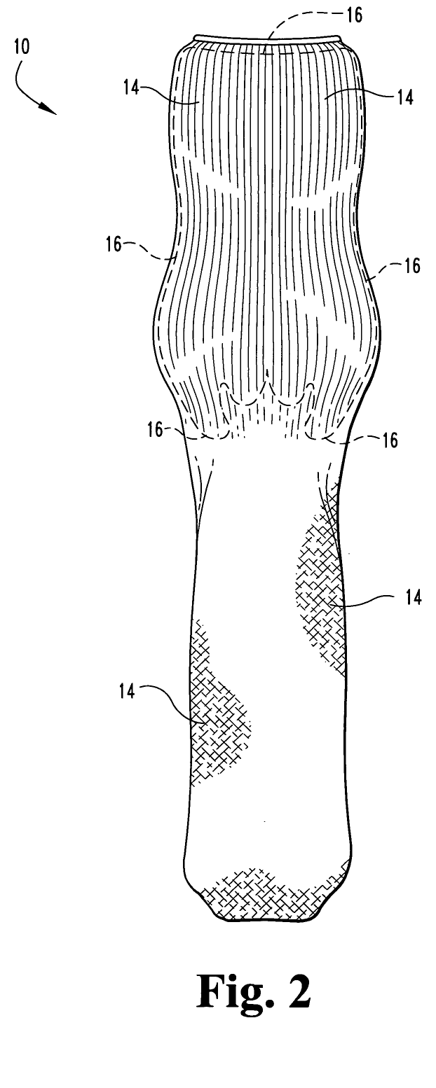

FIG. 3 is a front enlarged perspective view of a preferred elevating component of the present invention.

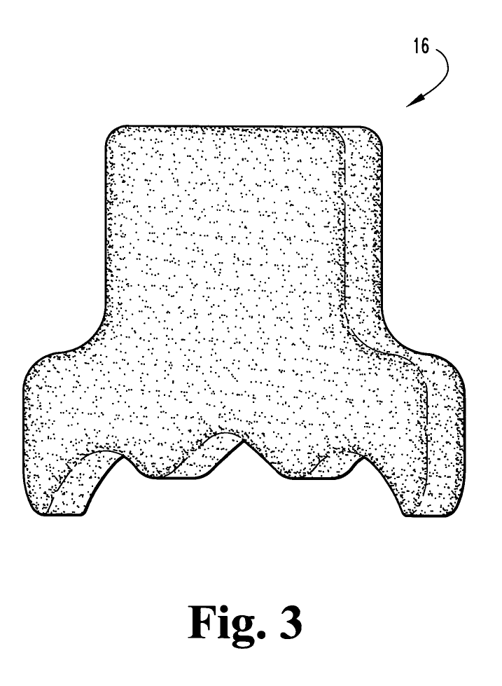

FIG. 4 is a left side elevational view of the therapeutic sock of FIG. 1 in place on a patient's foot.

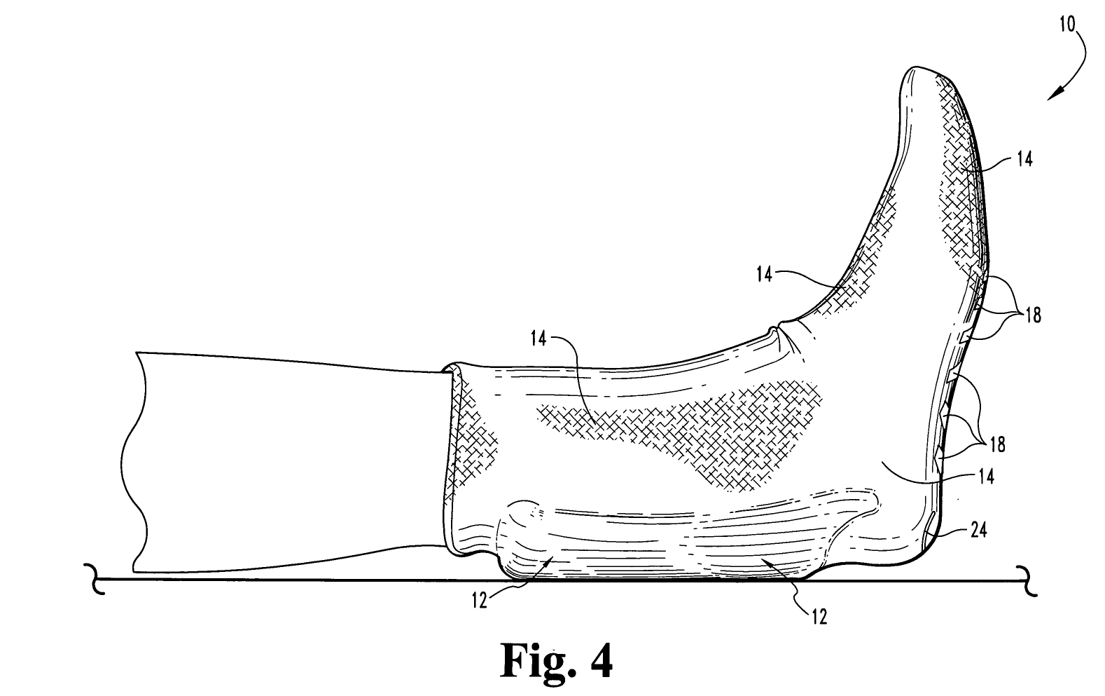

### DETAILED DESCRIPTION OF THE PREFERRED EMBODIMENT

The drawings only represent the preferred embodiment of the invention to date. The gist of the invention, in general terms, is the placement and attachment of an elevating component 12 within a sock 14. The preferred embodiment of the therapeutic sock 10 of the present invention is put on a patient's foot like a tube-sock 14. A generally “U” shaped foam insert 16 is attached to the inside of the tube-sock 14 to serve as the elevating component 12. The foam insert 16 is positioned so that when the therapeutic sock 10 is worn the foam insert 16 is placed on the wearer's Achilles tendon, which will lift the wearer's heel and relieve pressure caused by direct contact with any and all surfaces. In the preferred embodiment to date a line 24 is placed on the bottom of the therapeutic sock 10 to indicate where to position the point where a wearer's heel and Achilles tendon attach. A non-slip tread 18 covers the bottom of the tube-sock 14, allowing the wearer of the tube sock 14 to safely ambulate without removing the sock 14.

The elevating component 12 can be constructed from a variety of materials, including but not limited to foam, gel (flowing/nonflowing), air, soft fabric, or a combination of the above. The tube-sock material can be any type of fabric to allow for adaptation to the needs of a patient wearer (example: elastic, cotton, nylon, etc.) In the preferred embodiment to date the elevating component 12 has been placed in its own cloth pocket and the cloth pocket has been attached with stitches 22 within and to the tube-sock 14. The election of the location of the stitches 22 allows the foam to conform better to the rounded Achilles tendon.

The shape of the elevating component 12 is chosen so that it partially contours the calf and elevates the posterior heel, ankle and sides of the foot when in use. This design helps unload the soft tissue covering these bony areas and helps maintain the circulation to and from the region. The tube-sock 14 design along with an anti-slip head 18 allows the patient to wear the therapeutic sock 10 while in bed as well as while ambulating with assistance.

The design of the therapeutic sock 10 of the present invention will deliver a clinically and cost effective product that can be used by low risk patients who still have potential to develop pressure ulcers and other skin irritations in and about the heel, ankle and foot region.

1. A therapeutic sock, comprising
a sock having a first end to receive toes, a second end to overlay a calf, and a heel zone therebetween to overlay a heel; and
an elevating component shaped to contour the calf and to elevate the posterior heel, ankle and sides of a foot when in use, and positioned within and attached to the sock between the heel zone and the second end whereby when the sock is in use the elevating component is positioned over the Achilles tendon and relieves pressures caused by direct contacts with any and all surfaces.

2. The therapeutic sock of claim 1 wherein the sock is a tube-sock.

3. The therapeutic sock of claim 1 wherein the elevating component is generally “U” shaped foam.

4. The therapeutic sock of claim 2 wherein the tube-sock has anti-slip elements between the first end and the heel zone.

5. The therapeutic sock of claim 1 wherein the elevating component is attached to the sock by stitches whereby the elevating component bends thereabout to better conform to the Achilles tendon.
